(19) United States
(12) Patent Application Publication
(10) Pub. No.: US 2010/0168415 A1
(43) Pub. Date: Jul. 1, 2010

(54) Process for preparation of Caspofungin acetate

(76) Inventors: Kwang-Chung Lee, Su-Lin (TW); Yen-Shih Tung, Su-Lin (TW); Hao-Ling Fang, Su-Lin (TW)

Correspondence Address: Lee, Kwang-Chung, P. O. Box 55-846, Taipei, 104, TW

(21) Appl. No.: 12/653,914

(22) Filed: Dec. 22, 2009

(30) Foreign Application Priority Data
Dec. 31, 2008 (TW) 97151540

(51) Int. Cl. C07D 259/00

(52) U.S. Cl. 540/460

(57) ABSTRACT

A process for making caspofungin acetate comprising the steps of:
A. selectively dehydrating pneumocandin Bo to obtain a nitrile;
B. reducing the nitrile to primary amine;
C. reacting the primary amine with an arylthiol in a suitable solvent to obtain a thioether; and
D. reacting the thioether with ethylenediamine to obtain the caspofungin acetate having a formula as shown below:

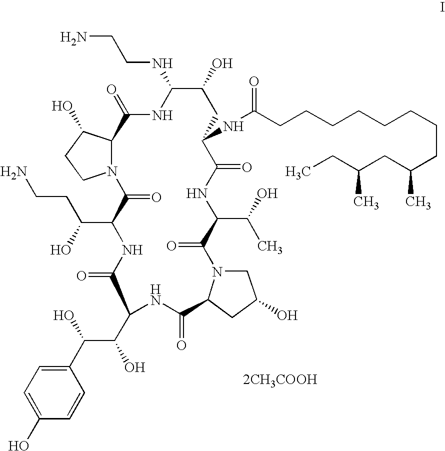

### BACKGROUND OF THE INVENTION

Caspofungin acetate is an aza cyclohexapeptide belonging to echinocandin family which is useful in treating systemic fungal infections, especially those caused by Candida, Aspergillus, Histoplasma, Coccidioides and Blastomyces.

There are several prior arts disclosing the preparation of Caspofungin acetate, but being unsatisfactory. U.S. Pat. No. 5,552,521 disclosed a process for preparing aza cyclohexapeptides including its Step 1 for the direct reduction of amide, Pneumocandin B₀, to a primary amine of its compound III, but only obtaining a yield of 47%. The unreacted amide is still remained as high as about 50% and should be further removed by a reverse phase column chromatography. Otherwise, the unreacted amide may affect the purity of Caspofungin acetate to thereby increase the difficulty of the purification and increase the production cost.

In the Step 2 of U.S. Pat. No. 5,552,521, its compound III reacted with thiophenol in the presence of trifluoroacetic acid to obtain its Compound IV. However, the thiophenol is a liquid compound with acute toxicity and unpleasant stinking smell, to thereby be harmful to the modern environmental protection.

U.S. Pat. No. 5,792,746 disclosed a 5 steps process for preparation of Caspofungin acetate. But the yield was even lower. U.S. Pat. Nos. 5,936,062 and 7,214,768 respectively disclosed processes similar to that of U.S. Pat. No. 5,552,521 by adding some boron compounds. However, these boron compounds are toxic and hazardous, unsuitable for mass production.

The present inventor has found the drawbacks of the prior arts, and invented the present process for preparation of Caspofungin acetate by overcoming the drawbacks of the prior arts.

### SUMMARY OF THE INVENTION

The object of the present invention is to provide a process for preparation of Caspofungin acetate comprising the steps of:
A. selectively dehydrating the amide group on Pneumocandin Bₒ to obtain a nitrile;
B. reducing the nitrile to obtain a primary amine;
C. reacting the amine with arylthiol in suitable solvent to obtain the thioether; and
D. reacting the thioether with ethylenediamine to obtain Caspofungin acetate.

### DETAILED DESCRIPTION

The process for Preparation of Caspofungin acetate, or aza cyclohexapeptide, of Compound I in accordance with the present invention comprises the steps as shown in the following reaction scheme:

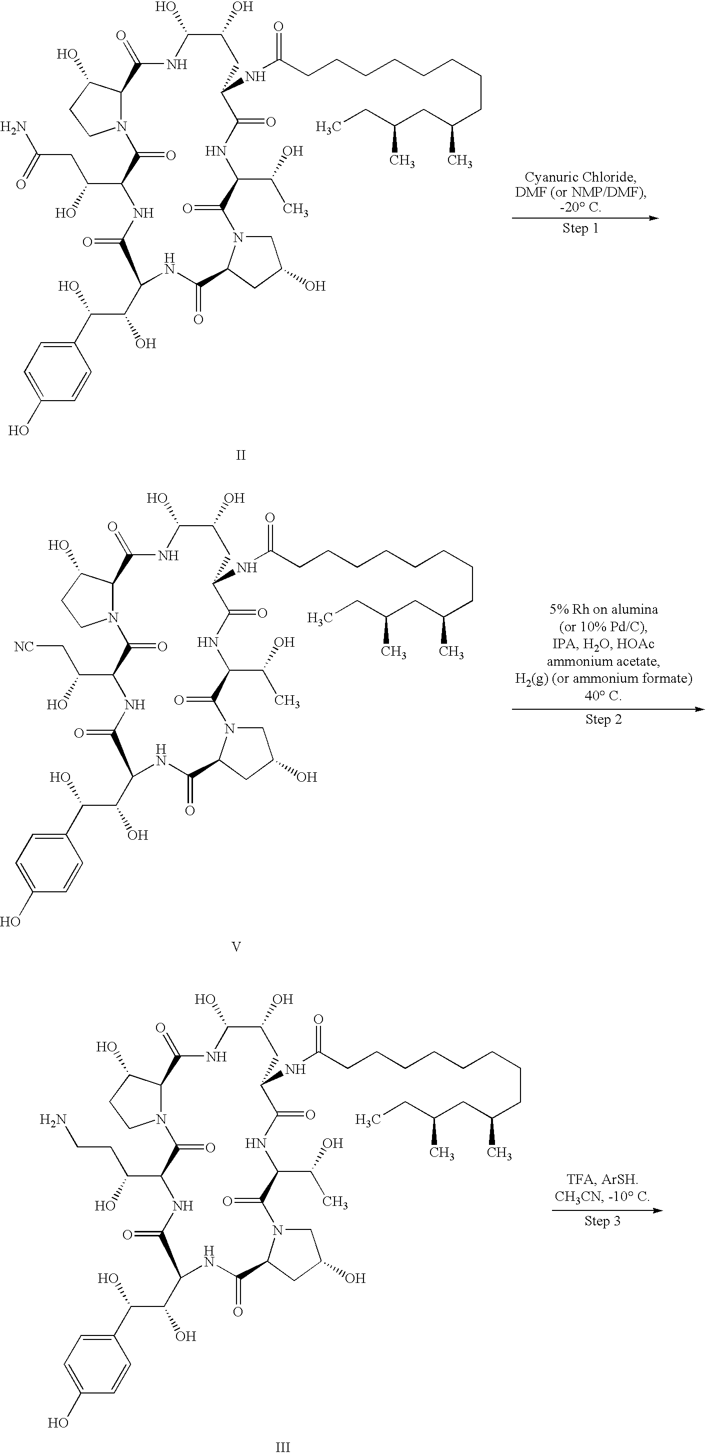

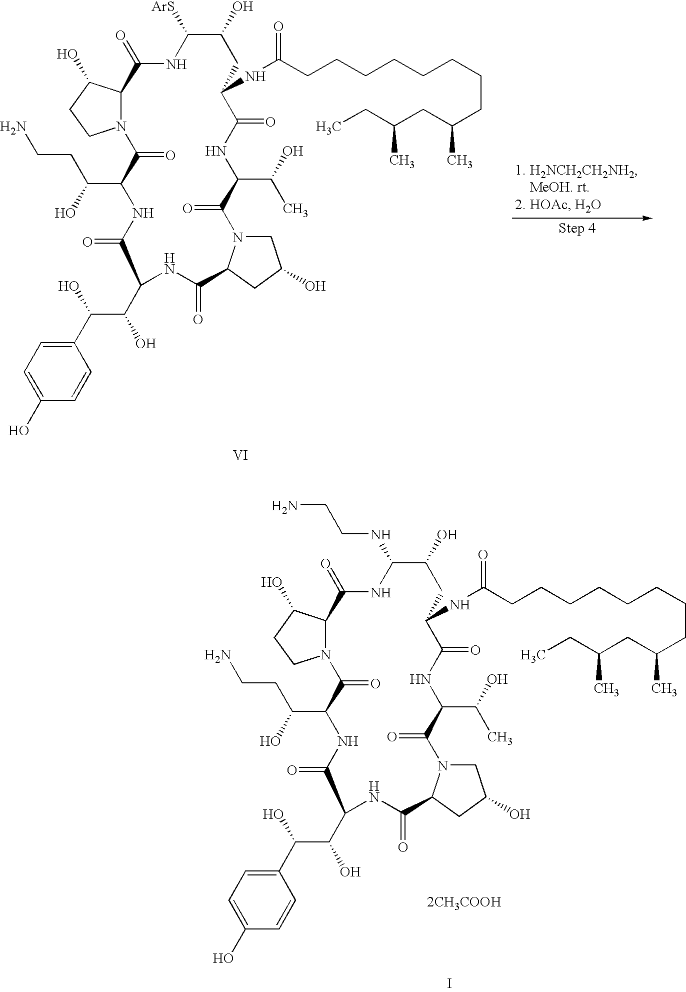

### Step 1:

For conducting a selective dehydration reaction, a starting reactant of pneumocandin Bₒ, the Compound II, is dissolved in suitable solvent. Cyanuric chloride is added to the resulting mixture at the temperature of −20° C. to obtain the nitrile with the formula of Compound V. The suitable solvent may be selected from N,N-Dimethylformamide (DMF), 1-methyl-2-pyrrolidone (NMP), etc. The reaction can be monitored by HPLC. The reaction may be stopped until the content of Pneumocandin Bₒ is less than 6% in area. It indicates a conversion rate is higher than 94%. Since the product is water insoluble, the product thus obtained by adding water therein, and then filtered, washed with water and dried to obtain white solid Compound V, having yield of 70˜75%.

### Step 2:

Compound V was reduced by catalytic hydrogenation with suitable catalyst. The hydrogen may be supplied such as from hydrogen gas cylinder, balloon or generated in situ from ammonium formate.

The catalytic reduction reaction is conducted at 40° C. in isopropyl alcohol (IPA), water, glacial acetic acid and ammonium acetate. The catalyst can be selected from 10% Pd/C or 5% Rh on alumina to convert the nitrile Compound V to a primary amine Compound III. It is monitored by HPLC analysis to indicate a conversion rate higher then 97%.

Then, the product mixtures were filtered by celite to remove Pd/C catalyst or other salts. The filtrate was diluted with water. Then the diluted solution was absorbed by RP-18 silica gel and desorbed by a mixture of methanol/acetonitrile/acetic acid (1:1:0.006 by volume). The product was collected and concentrated under vacuum. After evaporation, the product of Compound III was obtained, having a yield of 83-92%.

If the hydrogen was derived from ammonium formate. The above-mentioned step 2 may be repeated, except that the reaction was conducted at room temperature, and the catalyst was 10% Pd/C. After post treatment, Compound III was obtained, having a yield of 70%.

### Step 3:

The Compound III reacts with arylthiol in suitable solvent to obtain Compound VI. The arylthiol (ArSH) used in this step has a formula as below:

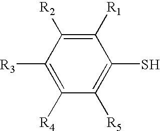

wherein, R₁, R₂, R₃, R₄, and R₅ is respectively selected from: hydrogen, alkyl group, methoxy, or halo substitutents.

The arylthiol may include: p-toluenethiol, thiophenol, o-toluenethiol, m-toluenethiol, benzylthiol, 4-methoxybenzenethiol, 4-Bromobenzenethiol, 4-Fluorobenzenethiol, 4-Chlorobenzenethiol, 4-Isopropyl benzenethiol, 4-tert-Butylbenzenethiol, 2,5-Dimethylbenzenethiol, 2,4-Dimethyl benzenethiol, 3,4-Dimethylbenzenethiol, 3-Ethoxythiophenol, and 2,4,6-Trimethylbenzene-1-thiol.

In this step, the p-toluenethiol is most preferable since it is a solid chemical to be easily handled by air cargo, and has lower toxicity and less stinky smell. So, it is very beneficial for environmental protection and better for human health, thereby being suitable for mass production.

In step 3, Compound III react with p-toluenethiol in the presence of trifluoroacetic acid (TFA) at −8° C.˜−10° C. to obtain Compound VI. The reaction product was monitored by HPLC, indicating a conversion rate higher then 97%. The reaction solution was diluted with water. The resulting solution was absorbed by RP-18 silica gel and desorbed by a mixture of methanol/trifluoroacetic acid (1:0.0005 by volume). The product was collected and concentrated under vacuum. After being dried, the product of Compound VI was obtained with a yield of 71%.

### Step 4:

The Compound VI react with ethylenediamine, with or without solvent to substitute the toluenethiol group in Compound VI. After completing such a substitution reaction, the reaction solution was diluted with water, The reaction solution was absorbed by RP-18 gel and desorbed by a mixture of methanol/acetonitrile/acetic acid (1:1:0.002 by volume). The product was collected and concentrated under vacuum. After drying, the product of Compound I was obtained.

In the substitution reaction, the suitable solvents may be selected from: methanol, ethanol, isopropyl alcohol, tetrahydrofuran, acetonitrile, N,N-Dimethylformamide and water.

The crude product was purified by reverse phase medium pressure column chromatography. It was eluted with a mixture of acetonitrile and 0.15% aqueous glacial acetic acid (1:4). The rich part was collected and lyophilized to obtain the white solid Compound I in di-acetate salt, having a purity of 98.57% analyzed by HPLC and a yield of 40%. The product Compound I was identified by ¹H-NMR and ¹³C-NMR spectra to be proven as Caspofungin Acetate as compared with the spectra shown in J. Org. Chem. 2007, 72, 2335-2343.

In a further embodiment, the present process for the preparation of Caspofungin acetate with high conversion rate in step 1, step2, step3 and step4. Especially in the first two steps compared with prior arts. In step 3, using a lower toxicity and less stinky smell of p-toluenethiol and without using the high toxic and hazard Boron compounds. It's only purified by MPLC in the last step.

Accordingly, the present invention is superior to the prior arts. The present invention may be further described in detail with reference to the examples as follows:

### Example 1

### Method 1:

The Compound II (Pneumocandin Bo, 2.0 g, 1.78 mmol) was dissolved in dry N,N-dimethylformamide (30 mL). The reaction was cooled to −17° C. and cyanuric chloride (1.68 g, 9.11 mmol) was added in one portion. The mixture was stirred at −20° C. until HPLC analysis showed <6 area % starting material (ca. 1 hr). Water (50 mL) was added over 10 minutes, and the mixture was warmed to 20° C. The mixture was slowly poured into vigorously stirred water (400 mL). The suspension was aged for 2 hours and then filtered. The product was thoroughly washed with water (200 mL) and then dried under vacuum. After drying, a white solid product of Compound V (1.73 g) was obtained, having a yield of 69%.

### Method 2:

The Compound II (Pneumocandin Bo, 59.7 g, 44.84 mmol) was dissolved in a mixture of dry 1-methyl-2-pyrrolidon (540 mL) and dry N,N-dimethylformamide (60 mL). The reaction mixture was cooled to −17° C. and cyanuric chloride (51.54 g, 280 mmol) was added in one portion. The mixture was stirred at −17° C. until HPLC analysis showed <6 area % starting material (ca. 4 hr). Water (300 mL) was added over 10 minutes, and the mixture was warmed to 20° C. The crude mixture was slowly poured into vigorously stirred water (7600 mL). The suspension was aged for 2 hr and then filtered. The product was thoroughly washed with water (2000 mL) and then dried under vacuum. After drying, a white solid product of Compound V (56.21 g) was obtained, having a yield of 75%.

### Example 2

### Method 1:

The Compound V (60 g, 31.5 mmol) was dissolved in a 85:15 mixture of 2-propanol/water (600 mL) and acetic acid (60 mL). Ammonium acetate (150 g, 1944 mmol) and 5% Rh on alumina catalyst (14 g) were added. The resulting mixture was hydrogenated with hydrogen balloon at 40° C. The reaction was stopped until HPLC analysis showed <1 area % of starting material (26 hr). The reaction solution was diluted with water to have a volume of 1500 mL. Then the catalyst was filtered off by celite. The filter cake was washed with water (500 mL). The reaction solution was diluted with water to have a volume of 24000 mL, then it was absorbed by RP-18 silica gel and desorbed by a mixture of methanol/acetonitrile/acetic acid (1:1:0.006 by volume). The product was collected and concentrated under vacuum. After drying, a yellow product of Compound III (56.3 g) was obtained, with a yield of 83%.

### Method 2:

The Compound V (1.5 g, 1.0 mmol) was dissolved in a 85:15 mixture of 2-propanol/water (15 mL) and acetic acid (1.95 mL). 10% Pd/C catalyst (1.5 g), and ammonium formate (12 g, 190 mmol) were added. The resulting mixture was stirred vigorously at room temperature for approximately 17 hours. The reaction was stopped until HPLC analysis showed <1 area % starting material. Then the catalyst was filtered off by celite. The filtered cake was washed with 2-propanol (50 mL). The reaction solution was diluted with water to have a volume of 500 mL, then it was absorbed by RP-18 silica gel and desorbed by a mixture of methanol/acetonitrile/trifluoroacetic acid (1:1:0.004 by volume). The product was collected and concentrated under vaccum. After drying, a yellow product of Compound III (1.46 g) was obtained, with a yield of 70%.

### Method 3:

The Compound V (60 g, 57.3 mmol) was dissolved in a 85:15 mixture of 2-propanol/water (600 mL) and acetic acid (60 mL). Ammonium acetate (150 g, 1944 mmol) and 10% Pd/C catalyst (24 g) were added. The resulting mixture was hydrogenated with hydrogen balloon at 40° C. The reaction was stopped until HPLC analysis showed <1 area % of starting material (28 hr). Then the catalyst was filtered off by celite. The filtered cake was washed with 2-propanol (800 mL). The resulting solution was concentrated under vacuum to a volume about 200 mL. The reaction solution was diluted with water to have a volume of 2000 mL, then it was absorbed by RP-18 silica gel and desorbed by a mixture of methanol/acetonitrile/acetic acid (1:1:0.006 by volume). The product was collected and concentrated under vacuum. After drying, a yellow product of Compound III (61.61 g) was obtained, with a yield of 92%.

### Example 3

Compound III (1.28 g, 0.66 mmol) was added to 40 mL anhydrous acetonitrile under nitrogen and cooled to less than −5° C., p-toluenethiol (786 mg, 6.33 mmol) was added. Trifluoroacetic acid (5.4 ml, 72.7 mmol) and acetonitrile (10 mL) were added over 30 minutes and kept the temperature of the reaction mixture below −5° C. The reaction was aged at −8° C.˜−10° C. until HPLC analysis showed <1 area % of starting material (20 hr). At this time, chilled water (50 mL) was slowly added into the reaction mixture in more than 30 minutes and kept the reaction mixture temperature below 0° C. The reaction was slowly warmed up to room temperature. The reaction solution was diluted with water. Then it was absorbed by RP-18 silica gel and desorbed by a mixture of methanol/trifluoroacetic acid (1:0.0005). The product is collected and concentrated under vacuum. After drying, a pale yellow solid product of Compound VI (1.101 g) was obtained, with a yield of 71%.

### Example 4

The solution of Compound VI (1.05 g, 0.44 mmole) in methanol (8 mL) was cooled to −10° C. Ethylenediamine (5 mL, 75 mmole) in methanol (4 mL). was added over 30 minutes and kept the temperature <5° C. This mixture was stirred for 45 hr at rt. The reaction was monitored by HPLC. At this time, a chilled 65% aqueous solution of glacial acetic acid (10 mL) was added slowly into the reaction mixture in more than 30 minutes and kept the reaction temperature below 0° C. Acetic acid was further added therein for adjusting its pH 5.6. Then, n-hexane (50 mL) was added to extract the water layer then separated. The organic layer was back-extracted with water and separated. Combined all the water layer. Then it was absorbed by RP-18 silica gel and desorbed by a mixture of methanol/acetonitrile/acetic acid (1:1:0.002 by volume). The product was collected and concentrated under vacuum, and dried to obtain a pale yellow solid, which was further purified by a Lichroprep RP-18 silica gel column (80.2 g) having a diameter of 26 mm, eluted by a solution of acetonitrile and 0.15% acetic acid (at a ratio of 1:4). The rich part was collected and lyophilized to obtain 196 mg of a white solid with a purity of 98.57% and a yield of 40%. It also was identified by ¹H-NMR spectrum.

The present invention may produce the aza cyclohexapeptide with high yields in each step of the process. Its purification is simple and convenient. The process is environmentally friendly since less toxic raw materials are used. So, the present invention is superior to the prior arts.

The present invention may be further modified without departing from the spirit and scope of this invention.

I claim:

1. A process for preparation of Compound I, Caspofungin acetate, having a formula as shown below: 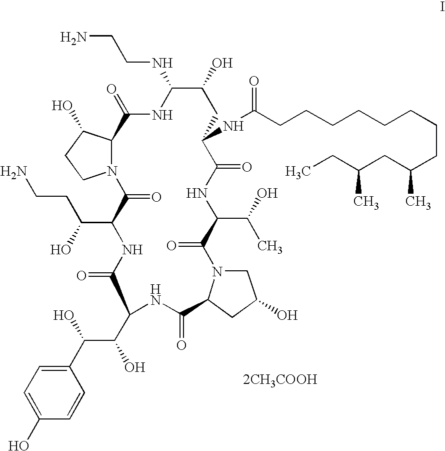
the process comprising the steps of:
A. Selectively dehydrating Pneumocandin Bo, Compound II, having a formula as shown below: 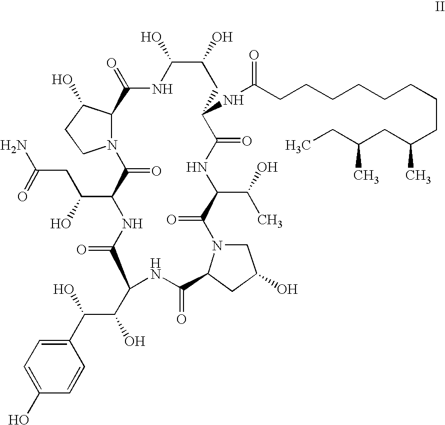
with a dehydration agent to obtain a nitrile with the formula of Compound V as shown below: 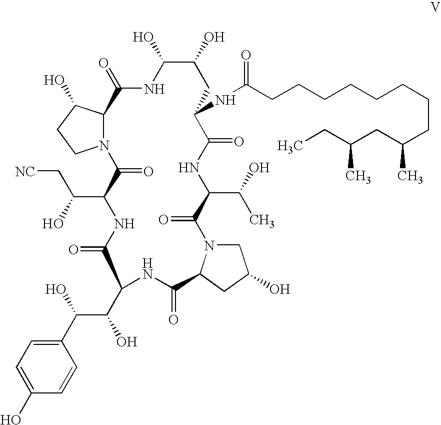
B. Reducing Compound V in the presence of a metal catalyst to obtain an amine with the formula of Compound III as shown below: 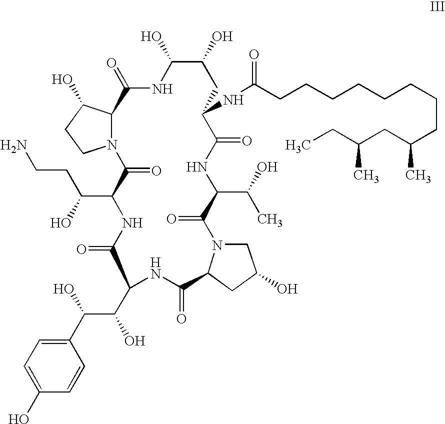
C. Reacting Compound III with an arylthiol in the presence of a solvent to obtain Compound VI as shown below: 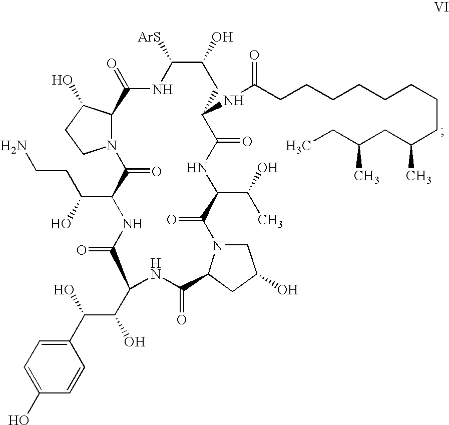
and
D. Reacting Compound VI with ethylenediamine in the presence of solvent or without solvent to obtain Caspofungin acetate of Compound I.

2. A process according to claim 1; wherein the dehydration agent as used in said step A is cyanuric chloride.

3. A process according to claim 2, wherein the step A further includes a solvent selected from the group consisting of: N,N-dimethylformamide (DMF), 1-methyl-2-pyrrolidone (NMP), and the mixture of said DMF and NMP, for dissolving Pneumocandin Bo in said solvent.

4. A process according to claim 1, wherein said step B is conducted with a hydrogen source selected from: hydrogen gas, and hydrogen generated from ammonium formate.

5. A process according to claim 1, wherein the metal catalyst as used in step B is selected from: Pd/C, Rh on alumina, and Rh/C.

6. A process according to claim 1, the arylthiol has a formula as indicated below: 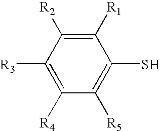
wherein R₁, R₂, R₃, R₄, and R₅ is respectively selected from the groups consisting of hydrogen, alkyl, methoxy, and halo substituents.

7. A process according to claim 6, wherein said arylthiol comprises: p-toluenethiol, thiophenol, o-toluenethiol, m-toluenethiol, benzylthiol, 4-methoxybenzenethiol, 4-Bromobenzenethiol, 4-Fluorobenzenethiol, 4-Chlorobenzenethiol, 4-Isopropyl benzenethiol, 4-tert-Butylbenzenethiol, 2,5-Dimethylbenzenethiol, 2,4-Dimethyl benzenethiol, 3,4-Dimethylbenzenethiol, 3-Ethoxythiophenol, and 2,4,6-Trimethylbenzene-1-thiol.

8. A process according to claim 1, wherein said solvent used in step C is acetonitrile.

9. A process according to claim 1, wherein said step D is conducted without solvent.

10. A process according to claim 1, wherein said solvent as used in step D is selected from the group consisting of: methanol, ethanol, isopropyl alcohol, tetrahydrofuran, dichloromethane, acetonitrile, N,N-dimethylformamide and water.
